(19) United States
(12) Patent Application Publication
(10) Pub. No.: US 2008/0171017 A1
(43) Pub. Date: Jul. 17, 2008

(54) Use Of Cd25 Antibodies In Immunotherapy

(76) Inventor: Andreas Katopodis, Bottmingen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/910,805

(22) PCT Filed: Apr. 13, 2006
(86) PCT No.: PCT/EP2006/003444

§ 371 Date: Oct. 5, 2007

(30) Foreign Application Priority Data
Apr. 15, 2005 (GB) 0507696.3

(51) Int. Cl. A61K 38/21; A61K 39/395; A61P 31/04; A61P 9/10; A61P 35/00

(52) U.S. Cl. 424/85.4; 424/133.1

(57) ABSTRACT

A method for the treatment of proliferative disease or infectious disease, comprising administering to the patient an effective amount of a CD25 binding molecule.

The present invention is directed to the use of a chimeric CD25 antibody, in particular basiliximab, in immunotherapy and more particularly in the prevention or treatment of a proliferative disease, such as cancer, or an infectious disease, such as microbial infection, wherein the inhibition of regulatory T cells is beneficial.

In view of the significant number of patients suffering from proliferative disease or infectious disease, in particular in the case of cancer, there is a need for an effective treatment against these diseases.

It is known that after activation T cells express the IL-2 receptor alpha chain (CD25) which is important for the proliferation of the activated T cells and the eventual clearing of the antigen. These antigen-activated, proliferating T cells are called herein effector T-cells.

In healthy state, a small percentage of the total T cell population expresses CD25 constitutively (without being activated). These cells have been shown to suppress the expansion of effector cells and are called regulatory T-cells. Regulatory T cells are important for the healthy state in order to keep effector T cells from reacting against self antigens or over-reacting to foreign antigens and damaging the organism. In a normal, protective immune response, effector T cells multiply after contact with foreign antigen and overcome inhibition by regulatory T cells thereby resulting in protection against infection or cancer. In case of proliferative disease, cancer cells and many infectious agents are able to evade the healthy immune response by increasing the amount of regulatory T cells and thereby limiting the generation of effector T cells against them.

In cases, such as transplantation, when immunosuppression is required, anti-CD25 monoclonal antibodies have been shown to provide a benefit by reducing the number of effector T cells.

It has now been found that anti-CD25 treatment is also useful for immunotherapy, i.e. for inhibiting, blocking or inactivating regulatory T-cells, e.g. the generation and/or multiplication of these cells. In particular it has been found that a chimeric CD25 antibody is useful specifically against diseases or disorders where inhibition of regulatory T cells is beneficial e.g. because regulatory T cells block an efficient function of effector T-cells and, such as certain proliferative diseases, e.g. certain cancers, or infectious diseases, e.g. microbial infections.

By “CD25 antibody” is meant antibody capable of binding to the CD25 antigen either alone or associated with other molecules to form high affinity IL-2 receptors which is present on regulatory T cells. For example CD25 antibody can be monoclonal antibody.

Since CD25 antigen is also present on the surface membrane of effector T-cells, CD25 antibody have a potential suppressive activity on the immune system. Therefore the use of CD25 antibody according to the invention should be essentially selective, i.e. the pool of effector T-cells should be kept intact while the category of regulatory T-cells should be inhibited.

By “chimeric CD25 antibody” is meant antibody which includes antigen-specificity encoding regions from a mouse with human sequences, in particular a non-human variable region fused to a human constant region. A preferred chimeric CD25 antibody according to the invention comprises only non-human sequences in the non-human variable region, e.g. only mouse sequences in the non-human variable region.

Accordingly in a first aspect, the present invention provides the use of a chimeric CD25 antibody in immunotherapy, e.g. in cancer immunotherapy, e.g. for preventing or treating proliferative and infectious diseases where the inhibition of regulatory T-cells is beneficial.

The present invention also provides the use of a chimeric CD25 antibody to inhibit, block or inactivate the regulatory T-cells, e.g. the generation and/or multiplication of these cells. In particular it is provided the use of a chimeric CD25 antibody specifically in diseases where regulatory T cells block an efficient function of effector T-cells.

The term “proliferative disease” includes malignant and non-malignant proliferative diseases, wherein regulator T-cells are involved, e.g. atherosclerosis, carcinomas and, tumors, infectious diseases, thrombosis, restenosis, sclerodermitis and fibrosis.

The term “cancer” as used herein includes, but is not limited to breast cancer; melanoma; epidermoid cancer; cancer of the colon and generally the GI tract, in particular gastric cancer, esophageal cancer; colorectal cancer; pancreas cancer; renal cell carcinoma; lung cancer, in particular small-cell lung cancer, and non-small-cell lung cancer; renal cell cancer; head and neck cancer; genitourinary cancer, e.g. cervical, uterine, ovarian, testicles, prostate or bladder cancer; Hodgkin's disease or Kaposi's sarcoma.

The term “tumor” encompasses liquid tumors and solid tumors.

By “solid tumors” are meant tumors and/or metastasis (wherever located) other than lymphatic cancer, e.g. brain and other central nervous system tumors (e.g. tumors of the meninges, brain, spinal cord, cranial nerves and other parts of central nervous system, e.g. glioblastomas or medulla blastomas); head and/or neck cancer; breast tumors; circulatory system tumors (e.g. heart, mediastinum and pleura, and other intrathoracic organs, vascular tumors and tumor-associated vascular tissue); excretory system tumors (e.g. kidney, renal pelvis, ureter, bladder, other and unspecified urinary organs); gastrointestinal tract tumors (e.g. oesophagus, stomach, small intestine, colon, colorectal, rectosigmoid junction, rectum, anus and anal canal), tumors involving the liver and intrahepatic bile ducts, gall bladder, other and unspecified parts of biliary tract, pancreas; oral cavity (lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx); reproductive system tumors (e.g. vulva, vagina, Cervix uteri, Corpus uteri, uterus, ovary, and other sites associated with female genital organs, placenta, penis, prostate, testis, and other sites associated with male genital organs); respiratory tract tumors (e.g. nasal cavity and middle ear, accessory sinuses, larynx, trachea, bronchus and lung, e.g. small cell lung cancer or non-small cell lung cancer); skeletal system tumors (e.g. bone and articular cartilage of limbs, bone articular cartilage and other sites); skin tumors (e.g. malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneum and peritoneum, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites.

Where hereinbefore and subsequently a tumor, a tumor disease, a carcinoma or a cancer is mentioned, also metastasis in the original organ or tissue and/or in any other location are implied alternatively or in addition, whatever the location of the tumor and/or metastasis is.

The term “infectious disease” as used herein refers, but is not limited, to microbial infection, such as the presence of bacteria. Such infectious include, for example, central nervous system infections, external ear infections, infections of the middle ear, such as acute otitis media, infections of the cranial sinuses, eye infections, infections of the oral cavity, such as infections of the teeth, gums and mucosa, upper respiratory tract infections, lower respiratory tract infections, genitourinary infections, gastrointestinal infections, gynecological infections, septicemia, bone and joint infections, skin and skin structure infections, bacterial endocarditis, burns, antibacterial prophylaxis of surgery, antibacterial prophylaxis in immunosuppressed patients, such as patients receiving cancer chemotherapy, or organ transplant patients and chronic diseases caused by infectious organisms, e.g. arteriosclerosis.

They can be caused by a variety of bacterial or prokaryotic organisms. Examples include, but are not limited to, Gram positive and Gram negative aerobic and anaerobic bacteria, including Staphylococci, e.g. S. aureus and S. epidermidis; Enterococci, e.g. E. faecalis and E. faecium; Streptococci, e.g. S. pneumoniae; Haemophilus, e.g. H. influenza; Moraxella, e.g. M. catarrhalis; Bacteroides, e.g., Bacteroides fragilis, Clostridium, e.g., Clostridium difficile, Niesseria, e.g., N. meningitidis and N. gonorrhoae, Legionella, and Escherichia, e.g. E. coli. Other examples include Mycobacteria, e.g. M. tuberculosis; intercellular microbes, e.g. Chlamydia and Rickettsiae; and Mycoplasma, e.g. M. pneumoniae; and Pseudomonas, e.g. P. aeruginosa; Helicobacter pylori; Helicobacter hepaticus and parasites, e.g. Plasmodium falciparum, Pneumonocystis carnii, Leishmania major, Schistosoma masoni, Candida albicans, Herpes simplex virus, human immunodeficiency virus, hepatitis C virus, cytomegalovirus.

In one aspect of the invention, the chimeric CD25 antibody comprises at least one antigen binding site comprising at least one domain which comprises in sequence, the hypervariable regions CDR1, CDR2 and CDR3; said CDR1 having the amino acid sequence Arg-Tyr-Trp-Met-His, said CDR2 having the amino acid sequence Ala-Ile-Tyr-Pro-Gly-Asn-Ser-Asp-Thr-Ser-Tyr-Asn-Gln-Lys-Phe-Glu-Gly, and said CDR3 having the amino acid sequence Asp-Tyr-Gly-Tyr-Tyr-Phe-Asp-Phe; or direct equivalents thereof in immunotherapy, e.g. in the treatment of proliferative disease or infectious disease.

Preferably a chimeric CD25 antibody is used comprising at least one antigen binding site comprising:

a) a first domain comprising in sequence the hypervariable regions CDR1, CDR2 and CDR3; said CDR1 having the amino acid sequence Arg-Tyr-Trp-Met-His, said CDR2 having the amino acid sequence Ala-Ile-Tyr-Pro-Gly-Asn-Ser-Asp-Thr-Ser-Tyr-Asn-Gln-Lys-Phe-Glu-Gly, and said CDR3 having the amino acid sequence Asp-Tyr-Gly-Tyr-Tyr-Phe-Asp-Phe and,
b) a second domain comprising in sequence the hypervariable regions CDR1′, CDR2′ and CDR3′, said CDR1′ having the amino acid sequence Ser-Ala-Ser-Ser-Ser-Ile-Ser-Tyr-Met-Gln, said CDR2′ having the amino acid sequence Asp-Thr-Ser-Lys-Leu-Ala-Ser, and said CDR3′ having the amino acid sequence His-Gln-Arg-Ser-Ser-Tyr-Thr; or direct equivalents thereof.

Unless otherwise indicated, any polypeptide chain is herein described as having an amino acid sequence starting at the N-terminal extremity and ending at the C-terminal extremity.

When the antigen binding site comprises both the first and second domains, these may be located on the same polypeptide molecule or, preferably, each domain may be on a different chain, the first domain being part of an immunoglobulin heavy chain or fragment thereof and the second domain being part of an immunoglobulin light chain or fragment thereof.

Accordingly, the invention also provides the use of a chimeric CD25 antibody which comprises at least one antigen binding site comprising either a first domain having an amino acid sequence identical or substantially identical to that shown in Seq. Id. No. 1 in EP 449,769B1, starting with amino acid at position 1 and ending with amino acid at position 117 or a first domain as described above and a second domain having an amino acid sequence identical or substantially identical to that shown in Seq. Id. No. 2 in EP 449,769B1, starting with amino acid at position 1 and ending with amino acid at position 104, the contents of EP 449,769B1 being herein incorporated by reference.

A more preferred chimeric CD25 antibody for use in accordance with the invention is selected from a chimeric anti-CD25 antibody which comprises at least

a) one immunoglobulin heavy chain or fragment thereof which comprises (i) a variable domain comprising in sequence the hypervariable regions CDR1, CDR2 and CDR3 and (ii) the constant part or fragment thereof of a human heavy chain; said CDR1 having the amino acid sequence Arg-Tyr-Trp-Met-His, said CDR2 having the amino acid sequence Ala-Ile-Tyr-Pro-Gly-Asn-Ser-Asp-Thr-Ser-Tyr-Asn-Gln-Lys-Phe-Glu-Gly, and said CDR3 having the amino acid sequence Asp-Tyr-Gly-Tyr-Tyr-Phe-Asp-Phe and
b) one immunoglobulin light chain or fragment thereof which comprises (i) a variable domain comprising in sequence the hypervariable regions CDR1′, CDR2′ and CDR3′ and (ii) the constant part or fragment thereof of a human light chain; said CDR1′ having the amino acid sequence Ser-Ala-Ser-Ser-Ser-Ile-Ser-Tyr-Met-Gln, said CDR2′ having the amino acid sequence Asp-Thr-Ser-Lys-Leu-Ala-Ser, and said CDR3′ having the amino acid sequence His-Gln-Arg-Ser-Ser-Tyr-Thr; and direct equivalents thereof.

Alternatively, a chimeric CD25 antibody for use in accordance with the invention may be selected from a single chain binding molecule which comprises an antigen binding site comprising

a) a first domain comprising in sequence the hypervariable regions CDR1, CDR2 and CDR3, said hypervariable regions having the amino acid sequences as shown in Seq. Id. No. 1 in EP 449,769B1, the contents of which is herein incorporated by reference,
b) a second domain comprising in sequence the hypervariable regions CDR1′, CDR2′ and CDR3′, said hypervariable regions having the amino acid sequences as shown in Seq. Id. No. 2 in EP 449,769B1, the contents of which is herein incorporated by reference, and
c) a peptide linker which is bound either to the N-terminal extremity of the first domain and to the C-terminal extremity of the second domain or to the C-terminal extremity of the first domain and to the N-terminal extremity of second domain; and direct equivalents thereof.

As it is well known, minor changes in an amino acid sequence such as deletion, addition or substitution of one, more or several amino acids may lead to an allelic form of the original protein which has identical or substantially identical properties, e.g. antigen binding properties. Thus, by the term “direct equivalents thereof” is meant either any single domain CD25 binding molecule (molecule X)

(i) in which the hypervariable regions CDR1, CDR2 and CDR3 taken as a whole are at least 80% homologous, preferably at least 90% homologous, more preferably at least 95% homologous to the hypervariable regions as shown in Seq. Id. No. 1 in EP 449,769B1 or FIGS. 3 and 4 of EP 451,216B1, the contents of which is herein incorporated by reference, and,
(ii) which is capable of inhibiting the binding of Interleukin 2 (IL-2) to its receptor substantially to the same extent as a reference molecule having framework regions identical to those of molecule X but having hypervariable regions CDR1, CDR2 and CDR3 identical to those shown in Seq. Id. No. 1 in EP 449,769B1 or FIGS. 3 and 4 of EP 451,216B1, the contents of which is herein incorporated by reference; or any CD25 binding molecule having at least two domains per binding site (molecule X′)
(i) in which the hypervariable regions CDR1, CDR2, CDR3, CDR1′, CDR2′ and CDR3′ taken as a whole are at least 80% homologous, preferably at least 90% homologous, more preferably at least 95% homologous to the hypervariable regions as shown in Seq. Id. No. 1 and 2 in EP 449,769B1, the content of which is herein incorporated by reference, and
(ii) which is capable of inhibiting the binding of IL-2 to its receptor substantially to the same extent as a reference molecule having framework regions and constant parts identical to molecule X′ but having hypervariable regions CDR1, CDR2, CDR3, CDR1′, CDR2′ and CDR3′ identical to those shown in Seq. Id. No. 1 and 2 in EP 449,769B1, the content of which is herein incorporated by reference.

This last criterion may be conveniently tested in various assays as described in EP 449,769B1, the content of which is herein incorporated by reference.

In one embodiment of the invention, the CD25 binding molecule is a chimeric CD25 antibody comprising at least

a) one heavy chain which comprises a variable domain having an amino acid sequence identical or substantially identical to that shown in Seq. Id. No. 1 in EP 449,769, the contents of which is herein incorporated by reference, starting with amino acid at position 1 and ending with amino acid at position 117 and the constant part of a human heavy chain; and
b) one light chain which comprises a variable domain having an amino acid sequence identical or substantially identical to that shown in Seq. Id. No. 2 in EP 449,769B1, starting with glutamic acid at position 1 and ending with glutamic acid at position 104 and the constant part of a human light chain, the contents of EP 449,769B1 being herein incorporated by reference.

The constant part of a human heavy chain may be of the γ1, γ2, γ3, γ4, μ, α1, α2, δ or ε type, preferably of the γ type, more preferably of the γ1 type, whereas the constant part of a human light chain may be of the κ or λ type (which includes the λ1, λ2 and λ3 subtypes) but is preferably of the κ type. The amino acid sequence of all these constant parts are given in Kabat et al., Sequences of Proteins of Immunological Interest, US Department of Health and Human Services, Public Health Service, NIH.

The most preferred CD25 antibody according to the invention is the chimeric antibody basiliximab which is commercially available as SIMULECT® from Novartis AG.

The CD25 binding molecules suitable for use in accordance with the present invention may be produced by techniques disclosed for example in EP 449,769B1, the content of which is herein incorporated by reference.

When the antigen binding site comprises both the first and second domains, these may be located on the same polypeptide molecule or, preferably, each domain may be on a different chain, the first domain being part of an immunoglobulin heavy chain or fragment thereof and the second domain being part of an immunoglobulin light chain or fragment thereof.

Accordingly, the invention also provides the use in immunotherapy of a CD25 binding molecule which comprises at least one antigen binding site comprising either a first domain having an amino acid sequence identical or substantially identical to that shown in Seq. Id. No. 1 in EP 449,769B1, starting with amino acid at position 1 and ending with amino acid at position 117 or a first domain as described above and a second domain having an amino acid sequence identical or substantially identical to that shown in Seq. Id. No. 2 in EP 449,769B1, starting with amino acid at position 1 and ending with amino acid at position 104, the contents of EP 449,769B1 being herein incorporated by reference.

The CD25 binding molecules suitable for use in accordance with the present invention may be produced by techniques disclosed for example in EP 449,769B1, the content of which is herein incorporated by reference.

The invention also provides
(i) A method of immunotherapy comprising administering a therapeutically effective amount of a chimeric CD25 antibody as described above to the patient in need of such a treatment.
(ii) A method for the prevention and treatment of proliferative or infectious disease, e.g. cancer or malignant disease, where the inhibition of regulatory T cells is beneficial in a patient in need thereof comprising administering to the patient a therapeutically effective amount of a chimeric CD25 antibody as described above, e.g. basiliximab.
(iii) A method for treating solid tumor invasiveness or symptoms associated with such tumor growth or for the prevention of the metastatic spread of tumors, e.g. solid tumors, or for the prevention of growth or development of micrometastases in a patient in need thereof comprising administering to the patient a therapeutically effective amount of a chimeric CD25 antibody as described above, e.g. basiliximab.
(iv) A method to treat, prevent, and/or reduce the severity of an infection or a proliferative disease, e.g. cancer or malignant disease, in which the inhibition of regulatory T cells is beneficial in a patient comprising administering to the patient a therapeutically effective amount of a chimeric CD25 antibody as described above, e.g. basiliximab.
(v) A method for enhancing the activity of a chemotherapeutic agent or for overcoming resistance to a chemotherapeutic agent in a subject in need thereof, comprising administering to said subject a therapeutically effective amount of a chimeric CD25 antibody as described above, e.g. basiliximab, either concomitantly or sequentially with said chemotherapeutic agent.
(vi) Use of a chimeric CD25 antibody as described above, e.g. basiliximab, in immunotherapy, in particular to inhibit, block or inactivate regulatory T-cells, for example to prevent or treat a proliferative or infectious disease where the inhibition of regulatory T cells is beneficial.
(vii) Use of a chimeric CD25 antibody as described above, e.g. basiliximab, for the preparation of a medicament for preventing or treating a proliferative or infectious disease where the inhibition of regulatory T cells is beneficial, for example the diseases or disorders mentioned hereinabove.
(viii) A pharmaceutical composition for use in a method as described in (i) to (v) which comprises a chimeric CD25 antibody as described above, e.g. basiliximab, and a pharmaceutically acceptable carrier or diluent therefor.
(ix) A chimeric CD25 antibody as described above, e.g. basiliximab, for use in the manufacture of a medicament for use in a method as described in (i) to (v).

For the use in accordance with the invention, the appropriate dosage of the chimeric CD25 antibody, e.g. basiliximab, will, of course, vary depending upon, for example, the particular CD25 antibody to be employed, the host, the mode of administration and the severity of the condition being treated and the effects desired. Satisfactory results are generally indicated to be obtained at dosages from about 0.1 mg to about 1000 mg, preferably from 1 to 100 mg, more preferably 20-50 mg. For example dosages can be 0.3 mg/kg body weight, 1 mg/kg body weight, 3 mg/kg body weight, 5 mg/kg body weight or 10 mg/kg body weight or within the range of 1-10 mg/kg. Administration may be in a single dose or in several doses over a period of time as long as may be indicated in relation to the time the disease is clinically evident or prophylactically to suppress further clinical relapse, for example a dose from about 5 up to about 100 mg, may be administered once a month, until control or amelioration of the disease is achieved. A preferred dosage regimen comprises administration of 20-50 mg of chimeric CD25 antibody, e.g. basiliximab, every two weeks or once a month. An exemplary treatment regime entails administration once per week, once every two weeks, once every three weeks, once every four weeks, once a month, once every 3 months or once every three to 6 months. The chimeric CD25 antibody is conveniently administered parenterally, e.g. intravenously, for example, into the antecubital or other peripheral vein. An alternative exemplary dosing regimen is intravenous administration of e.g. 40 mg every month, e.g. every 28 days, until control or amelioration of the disease is achieved. The dosage regimens for the chimeric CD25 antibody of the invention may include every four weeks for six dosages, then every three months; every three weeks; 3 mg/kg body weight once followed by 1 mg/kg body weight every three weeks.

The chimeric CD25 antibody, e.g. basiliximab, is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

The intravenous infusions may be prepared as follows: the lyophylized antibodies are mixed together and dispersed into 100 ml sterile buffered saline containing 4.5% wt. of human albumin. This saline dispersion may be administered to the patients either as an intravenous bolus injection or as an intravenous infusion over a 15 minute period.

Investigations so far indicate that the administration of the chimeric CD25 antibody is free from unacceptable side-effects at the dosage levels employed. Particularly the preferred one, basiliximab, is safe, approved by the Federal Drug Administration (FDA) of the United States and are commercially available.

Pharmaceutical compositions of the invention may be manufactured in a conventional manner as described, e.g. in EP 449,769B1, the content of which is herein incorporated by reference.

The chimeric CD25 antibody of the invention may be administered as the sole active ingredient or together with other drugs in immunotherapy regimens or other anti-proliferative or chemotherapeutic agents or anti-infectious agents. For example, the chimeric CD25 antibody may be in combination with pharmaceutical compositions effective in various diseases as described above, e.g. with an mTOR inhibitor, e.g. rapamycin or rapamycin derivative, aromatase inhibitor, antiestrogen, anti-androgen, gonadorelin agonist, topoisomerase I or topoisomerase II inhibitor, microtubule active agent, alkylating agent, antineoplastic antimetabolite or platin compound, compound targeting/decreasing a protein or lipid kinase activity or a protein or lipid phosphatase activity, anti-angiogenic compound, compound which induces cell differentiation processes, bradykinin 1 receptor or an angiotensin II antagonist, cyclooxygenase inhibitor, bisphosphonate, histone deacetylase inhibitor, heparanase inhibitor (prevents heparan sulphate degradation), e.g. PI-88, biological response modifier, preferably a lymphokine or interferons, e.g. interferon γ, ubiquitination inhibitor, or inhibitor which blocks anti-apoptotic pathways, inhibitor of Ras oncogenic isoforms, e.g. H-Ras, K-Ras or N-Ras, or farnesyl transferase inhibitor, e.g. L-744,832 or DK8G557, telomerase inhibitor, e.g. telomestatin, protease inhibitor, matrix metalloproteinase inhibitor, methionine aminopeptidase inhibitor, e.g. bengamide or a derivative thereof, or proteosome inhibitor, e.g. PS-341.

The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN™. Formestane can be administered, e.g., in the form as it is marketed, e.g. under the trademark LENTARON™. Fadrozole can be administered, e.g., in the form as it is marketed, e.g. under the trademark AFEMA™. Anastrozole can be administered, e.g., in the form as it is marketed, e.g. under the trademark ARIMIDEX™. Letrozole can be administered, e.g., in the form as it is marketed, e.g. under the trademark FEMARA™ or FEMAR™ Aminoglutethimide can be administered, e.g., in the form as it is marketed, e.g. under the trademark ORIMETEN™. A combination of the invention comprising a chemotherapeutic agent which is an aromatase inhibitor is particularly useful for the treatment of hormone receptor positive tumors, e.g. breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX™. Raloxifene hydrochloride can be administered, e.g., in the form as it is marketed, e.g. under the trademark EVISTA™. Fulvestrant can be formulated as disclosed in U.S. Pat. No. 4,659,516 or it can be administered, e.g., in the form as it is marketed, e.g. under the trademark FASLODEX™. A combination of the invention comprising a chemotherapeutic agent which is an antiestrogen is particularly useful for the treatment of estrogen receptor positive tumors, e.g. breast tumors.

The term “anti-androgen” as used herein relates to any substance which is capable of inhibiting the biological effects of androgenic hormones and includes, but is not limited to, bicalutamide (CASODEX™), which can be formulated, e.g. as disclosed in U.S. Pat. No. 4,636,505.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX™. Abarelix can be formulated, eg. as disclosed in U.S. Pat. No. 5,843,901.

The term “topoisomerase I inhibitor” as used herein includes, but is not limited to topotecan, irinotecan, 9-nitrocamptothecin and the macromolecular camptothecin conjugate PNU-166148 (compound A1 in WO99/17804). Irinotecan can be administered, e.g. in the form as it is marketed, e.g. under the trademark CAMPTOSAR™. Topotecan can be administered, e.g., in the form as it is marketed, e.g. under the trademark HYCAMTIN™.

The term “topoisomerase II inhibitor” as used herein includes, but is not limited to the anthracyclines such as doxorubicin (including liposomal formulation, e.g. CAELYX™), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS™. Teniposide can be administered, e.g. in the form as it is marketed, e.g. under the trademark VM 26-BRISTOL™ Doxorubicin can be administered, e.g. in the form as it is marketed, e.g. under the trademark ADRIBLASTIN™. Epirubicin can be administered, e.g. in the form as it is marketed, e.g. under the trademark FARMORUBICIN™. Idarubicin can be administered, e.g. in the form as it is marketed, e.g. under the trademark ZAVEDOS™. Mitoxantrone can be administered, e.g. in the form as it is marketed, e.g. under the trademark NOVANTRON™.

The term “microtubule active agent” relates to microtubule stabilizing and microtubule destabilizing agents including, but not limited to taxanes, e.g. paclitaxel and docetaxel, vinca alkaloids, e.g., vinblastine, especially vinblastine sulfate, vincristine especially vincristine sulfate, and vinorelbine, discodermolides and epothilones and derivatives thereof, e.g. epothilone B or a derivative thereof. Paclitaxel may be administered e.g. in the form as it is marketed, e.g. TAXOL™. Docetaxel can be administered, e.g., in the form as it is marketed, e.g. under the trademark TAXOTERE™. Vinblastine sulfate can be administered, e.g., in the form as it is marketed, e.g. under the trademark VINBLASTIN R.P.™. Vincristine sulfate can be administered, e.g., in the form as it is marketed, e.g. under the trademark FARMISTIN™. Discodermolide can be obtained, e.g., as disclosed in U.S. Pat. No. 5,010,099.

The term “alkylating agent” as used herein includes, but is not limited to cyclophosphamide, ifosfamide, melphalan or nitrosourea (BCNU or Gliadel™). Cyclophosphamide can be administered, e.g., in the form as it is marketed, e.g. under the trademark CYCLOSTIN™. Ifosfamide can be administered, e.g., in the form as it is marketed, e.g. under the trademark HOLOXAN™.

The term “antineoplastic antimetabolite” includes, but is not limited to 5-fluorouracil, capecitabine, gemcitabine, methotrexate, hydrocloroquine, sulfasalazine and edatrexate. Capecitabine can be administered, e.g., in the form as it is marketed, e.g. under the trademark XELODA™. Gemcitabine can be administered, e.g., in the form as it is marketed, e.g. under the trademark GEMZAR™.

The term “platin compound” as used herein includes, but is not limited to carboplatin, cis-platin and oxaliplatin. Carboplatin can be administered, e.g., in the form as it is marketed, e.g. under the trademark CARBOPLAT™. Oxaliplatin can be administered, e.g., in the form as it is marketed, e.g. under the trademark ELOXATIN™. The term “compounds targeting/decreasing a protein or lipid kinase activity or further anti-angiogenic compounds” as used herein includes, but is not limited to protein tyrosine kinase and/or serine and/or threonine kinase inhibitors or lipid kinase inhibitors, e.g. compounds targeting, decreasing or inhibiting the activity of the epidermal growth factor family of receptor tyrosine kinases (EGFR, ErbB2, ErbB3, ErbB4 as homo- or heterodimers), the vascular endothelial growth factor family of receptor tyrosine kinases (VEGFR), the platelet-derived growth factor-receptors (PDGFR), the fibroblast growth factor-receptors (FGFR), the insulin-like growth factor receptor 1 (IGF-1R), the Trk receptor tyrosine kinase family, the Axl receptor tyrosine kinase family, the Ret receptor tyrosine kinase, the Kit/SCFR receptor tyrosine kinase, members of the c-Abl family and their gene-fusion products (e.g. BCR-Abl), members of the protein kinase C(PKC) and Raf family of serine/threonine kinases, members of the MEK, SRC, JAK, FAK, PDK or PI(3) kinase family, or of the PI(3)-kinase-related kinase family, and/or members of the cyclin-dependent kinase family (CDK) and anti-angiogenic compounds having another mechanism for their activity, e.g. unrelated to protein or lipid kinase inhibition.

Compounds which target, decrease or inhibit the activity of VEGFR are especially compounds, proteins or antibodies which inhibit the VEGF receptor tyrosine kinase, inhibit a VEGF receptor or bind to VEGF, and are in particular those compounds, proteins or monoclonal antibodies generically and specifically disclosed in WO 98/35958, e.g. 1-(4 chloroanilino)-4-(4-pyridylmethyl)phthalazine or a pharmaceutically acceptable salt thereof, e.g. the succinate, or in WO 00/09495, WO 00/27820, WO 00/59509, WO 98/11223, WO 00/27819 and EP 0 769 947; those as described by M. Prewett et al in Cancer Research 59 (1999) 5209-5218, by F. Yuan et al in Proc. Natl. Acad. Sci. USA, vol. 93, pp. 14765-14770, Dec. 1996, by Z. Zhu et al in Cancer Res. 58, 1998, 3209-3214, and by J. Mordenti et al in Toxicologic Pathology, Vol. 27, no. 1, pp 14-21, 1999; in WO 00/37502 and WO 94/10202; Angiostatin™, described by M. S. O'Reilly et al, Cell 79, 1994, 315-328; Endostatin™, described by M. S. O'Reilly et al, Cell 88, 1997, 277-285; anthranilic acid amides; ZD4190; ZD6474; SU5416; SU6668; or anti-VEGF antibodies or anti-VEGF receptor antibodies,e.g. RhuMab.

By antibody is meant intact monoclonal antibodies, polyclonal antibodies, multispecific antibodies formed from at least 2 intact antibodies, and antibodies fragments so long as they exhibit the desired biological activity.

Compounds which target, decrease or inhibit the activity of the epidermal growth factor receptor family are especially compounds, proteins or antibodies which inhibit members of the EGF receptor tyrosine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (Herpetin®), cetuximab, Iressa, OSI-774, CI-1033, EKB-569, GW-2016, E1.1, E2.4, E2.5, E6.2, E6.4, E2.11, E6.3 or E7.6.3. Compounds which target, decrease or inhibit the activity of PDGFR are especially compounds which inhibit the PDGF receptor, e.g. a N-phenyl-2-pyrimidine-amine derivative, e.g. imatinib.

Compounds which target, decrease or inhibit the activity of c-Abl family members and their gene fusion products, e.g. a N-phenyl-2-pyrimidine-amine derivative, e.g. imatinib; PD180970; AG957; or NSC 680410.

Compounds which target, decrease or inhibit the activity of protein kinase C, Raf, MEK, SRC, JAK, FAK and PDK family members, or PI(3) kinase or PI(3) kinase-related family members, and/or members of the cyclin-dependent kinase family (CDK) are especially those staurosporine derivatives disclosed in EP 0 296 110, e.g. midostaurin; examples of further compounds include e.g. UCN-01, safingol, BAY 43-9006, Bryostatin 1, Perifosine; Ilmofosine; RO 318220 and RO 320432; GO 6976; Isis 3521; or LY333531/LY379196.

Further anti-angiogenic compounds are e.g. thalidomide (THALOMID) and TNP-470.

Compounds which target, decrease or inhibit the activity of a protein or lipid phosphatase are e.g. inhibitors of phosphatase 1, phosphatase 2A, PTEN or CDC25, e.g. okadaic acid or a derivative thereof.

Compounds which induce cell differentiation processes are e.g. retinoic acid, α-, γ- or δ-tocopherol or α-, γ- or δ-tocotrienol.

The term cyclooxygenase inhibitor as used herein includes, but is not limited to, e.g. celecoxib (Celebrex®), rofecoxib (Vioxx®), etoricoxib, valdecoxib or a 5-alkyl-2-arylaminophenylacetic acid, e.g. 5-methyl-2-(2′-chloro-6′-fluoroanilino)phenyl acetic acid.

The term “histone deacetylase inhibitor” as used herein includes, but is not limited to MS-27-275, SAHA, pyroxamide, FR-901228 or valproic acid.

The term “bisphosphonates” as used herein includes, but is not limited to, etridonic, clodronic, tiludronic, pamidronic, alendronic, ibandronic, risedronic and zoledronic acid. “Etridonic acid” can be administered, e.g., in the form as it is marketed, e.g. under the trademark DIDRONEL™. “Clodronic acid” can be administered, e.g., in the form as it is marketed, e.g. under the trademark BONEFOS™. “Tiludronic acid” can be administered, e.g., in the form as it is marketed, e.g. under the trademark SKELID™. “Pamidronic acid” can be administered, e.g. in the form as it is marketed, e.g. under the trademark AREDIA™. “Alendronic acid” can be administered, e.g., in the form as it is marketed, e.g. under the trademark FOSAMAX™. “Ibandronic acid” can be administered, e.g., in the form as it is marketed, e.g. under the trademark BONDRANAT™. “Risedronic acid” can be administered, e.g., in the form as it is marketed, e.g. under the trademark ACTONEL™. “Zoledronic acid” can be administered, e.g. in the form as it is marketed, e.g. under the trademark ZOMETA™

The term “matrix metalloproteinase inhibitor” as used herein includes, but is not limited to collagen peptidomimetic and nonpetidomimetic inhibitors, tetracycline derivatives, e.g. hydroxamate peptidomimetic inhibitor batimastat and its orally bioavailable analogue marimastat, prinomastat, BMS-279251, BAY 12-9566, TAA211 or AAJ996.

In addition to therapies meant to reduce the growth of cancer cells, the chimeric CD25 antibody may be used in combination with therapies intended to boost the patient's immune system. These include, but are not limited to, IL-12, IL-15, IL-21, activated dendritic cells, various forms of vaccination (including DNA vaccination), IFN-α and other treatments intended to increase the function of effector T cells against the tumor or infectious agent.

The chimeric CD25 antibody, e.g. basiliximab, can also be applied in combination with surgical intervention, mild prolonged whole body hyperthermia and/or irradiation therapy.

The chimeric CD25 antibody, e.g. basiliximab, may also be administered together with other drugs effective in infectious diseases, such as antibiotics, antibacterial agents or antiviral compounds, such as β-lactams e.g. penicillins; cephalosporins; carbapenems; ketolides; quinolones e.g. fluoroquinolones; macrolides e.g. clarithromycin, azithromycin or vancomycin; rifamycins; monobactams; isoniazid; licosamides; mupirocin; sulfonamides; phenicols; fosfomycin; glycopeptides; tetracyclines; streptogramins; chloramphenicol; and oxazolidinone, famciclovir or penciclovir

Accordingly, the present invention also provides
(x) A method of immunotherapy in a patient comprising administering, e.g. concomitantly or in sequence, to said subject a therapeutically effective amount of a) a chimeric CD25 antibody as described above, e.g. basiliximab, and b) a further drug substance effective in immunotherapy, e.g. effective in inhibiting or inactivating regulator T-cells, e.g. an antigen binding molecule to at least one antigen other than CD25 which is present on regulator T-cells, or in increasing the function of effector T cells to the patient in need of such a treatment.
(xi) A method for the prevention and treatment of proliferative disease or infectious disease in a patient comprising administering, e.g. concomitantly or in sequence, to said subject a therapeutically effective amount of a) a chimeric CD25 antibody as described above, e.g. basiliximab, and b) a further drug substance being effective in the prevention or treatment of proliferative disease or infectious disease, e.g. in the prevention or treatment of atherosclerosis, carcinomas, thrombosis, restenosis, sclerodermitis, fibrosis or a cancer, malignant disease, e.g. solid tumor, e.g. as described above.
(xii) A method for the prevention and treatment of proliferative disease or infectious disease in a patient comprising administering, e.g. concomitantly or in sequence, to said subject an effective amount of a) a chimeric CD25 antibody as described above, e.g. basiliximab, and b) a composition comprising an anti-proliferative agent, a chemotherapeutic agent or an anti-infectious agent, e.g. an agent effective in the prevention or treatment of proliferative disease or infectious disease, e.g. in the prevention or treatment of atherosclerosis, carcinomas, thrombosis, restenosis, sclerodermitis, fibrosis or a cancer, malignant disease, e.g. solid tumor, e.g. as described above.
(xiii) A combination, e.g. a pharmaceutical kit or package, comprising a) a chimeric CD25 antibody, e.g. basiliximab, and b) a further drug substance effective in immunotherapy, e.g. effective in inhibiting or inactivating regulator T-cells, e.g. an antigen binding molecule to at least one antigen other than CD25 which is present on regulator T-cells, or in increasing the function of effector T-cells to the patient in need of such a treatment.
(xiv) A combination, e.g. a pharmaceutical kit or package, comprising a) a chimeric CD25 antibody as described above, e.g. basiliximab, and b) another drug substance effective in treating or preventing proliferative disease, such as cancer or tumor disease, e.g. solid tumor, or in treating or preventing infectious disease, such as a microbial infection.
(xv) The use of a combination as described in paragraph (xiii) or (xiv) below for immunotherapy or for use in any of the methods as described in paragraph (i) to (v) below, e.g. for the delay of progression or treatment of a proliferative disease, especially a tumor disease; or infectious disease, or for enhancing the activity of a chemotherapeutic agent or for overcoming resistance to a chemotherapeutic agent.
(xvi) A pharmaceutical composition comprising a combination as described in paragraph (xiii) or (xiv) below.
(xvii) A method as described in paragraph (i) to (v) or (x) to (xii) below, wherein the chimeric CD25 antibody preferably basiliximab, is administered intermittently.

If the chimeric CD25 antibody is co-administered with a further drug substance both may be packaged separately within the same container, with instructions for mixing or concomitant administration. Examples of kits include for example a multi-barreled syringe or a twin pack containing separate unit dose forms.

Utility of the chimeric CD25 antibody in treating e.g. solid tumors as hereinabove specified, may be demonstrated in animal test methods as well as in clinic, for example in accordance with the methods hereinafter described.

### A. In Vivo: Activity in Ca20948 Rat Pancreatic Tumors

Tumors are established in male Lewis rats by subcutaneous injection of CA20948 tumor cell suspension derived from donor rats into the left flank. Treatment is started on day 4 post inoculation. The chimeric CD25 antibody to be tested is administered p.o. once per day (or once every 2-4 days) from day 4 to day 9-15 post inoculation. Antitumor activity is expressed as T/C % (mean increase in tumor volumes of treated animals divided by the mean increase of tumor volumes of control animals multiplied by 100) and % regressions (tumor volume minus initial tumor volume divided by the initial tumor volume and multiplied by 100).

### B. Clinical Trial

Utility of the CD25 antibody of the invention in immunotherapy, e.g. cancer immunotherapy, can be shown by using a Basiliximab to study the effects on cancer progression or regression of infection. The following parameters may be assessed at baseline and after several weeks or months of treatment, e.g. after 2 or 3 months: CD25+ regulatory T cells in circulation or in the target organ, immune response against the cancer or infectious agent, other parameters relating to the effects of malignancies or infectious agents such as size, frequency, viral load, etc.

For example the use of CD25 antibody in cancer immunotherapy can be assessed with the following clinical example which describes the use of basiliximab in the maintenance of remission of colorectal cancer.

60 colorenal cancer patients are enrolled for the trial. Eligible criteria include age of at least 18 years, histologically confirmed metastatic colorectal carcinoma, with bidimensionally measurable disease. The patients are randomized to receive either standard cancer treatment or standard cancer treatment plus 1 to 10 mg/kg of basiliximab every two weeks.

The primary evidence of efficacy is based on objective response rates, and the time to response, duration of response, time to treatment failure and survival are also evaluated. Tumors are required to be measurable in at least one site of disease, and response characterization based on Southwestern Oncology Group (SWOG) criteria. After the baseline evaluation, tumor status can be assessed every 6 weeks for the first 24 weeks of the study and then every 12 weeks for the remainder of therapy. All complete and partial responses may require confirmation at least four weeks after they are first noted.

Basiliximab can be administered concomitantly with chemotherapy.

1. A pharmaceutical combination comprising: a) a chimeric CD25 antibody; and b) at least one anti-proliferative agent, chemotherapeutic agent or anti-infectious agent.

2. A combination according to claim 1, wherein the chimeric CD25 antibody comprises at least one antigen binding site comprising at least one domain that comprises in sequence, the hypervariable regions CDR1, CDR2 and CDR3; said CDR1 having the amino acid sequence Arg-Tyr-Trp-Met-His, said CDR2 having the amino acid sequence Ala-Ile-Tyr-Pro-Gly-Asn-Ser-Asp-Thr-Ser-Tyr-Asn-Gln-Lys-Phe-Glu-Gly, and said CDR3 having the amino acid sequence Asp-Tyr-Gly-Tyr-Tyr-Phe-Asp-Phe; or direct equivalents thereof.

3. A combination according to claim 2, wherein the chimeric CD25 antibody is basiliximab.

4. A combination according to claim 1, wherein the component b) is a chemotherapeutic agent selected from
i. an aromatase inhibitor,
ii. an antiestrogen, an anti-androgen or a gonadorelin agonist,
iii. a topoisomerase I inhibitor or a topoisomerase II inhibitor,
iv. a microtubule active agent, an alkylating agent, an antineoplastic antimetabolite or a platin compound,
v. a compound targeting/decreasing a protein or lipid kinase activity or a protein or lipid phosphatase activity, a further anti-angiogenic compound or a compound which induces cell differentiation processes,
vi. a bradykinin 1 receptor or an angiotensin II antagonist,
vii. a cyclooxygenase inhibitor, a bisphosphonate, a histone deacetylase inhibitor, a heparanase inhibitor (that prevents heparan sulphate degradation, a biological response modifier an ubiquitination inhibitor, or an inhibitor which blocks anti-apoptotic pathways,
viii. an inhibitor of Ras oncogenic isoforms or a farnesyl transferase inhibitor,
ix. a telomerase inhibitor,
x. a protease inhibitor, a matrix metalloproteinase inhibitor, a methionine aminopeptidase inhibitor, or a proteosome inhibitor or
xi. an mTOR inhibitor.

5. A combination according to claim 1 for use in immunotherapy.

6. A medicament or a kit for treating or preventing a proliferative disease or an infectious disease, comprising a combination according to claim 1, wherein inhibition of regulatory T cells is beneficial for the treatment or prevention of the proliferative or infectious disease.

7. A method of immunotherapy comprising administering an effective amount of basiliximab to a patient in need thereof.

8. A method of preventing or treating atherosclerosis, carcinomas, thrombosis, restenosis, sclerodermitis, fibrosis or for treating solid tumor invasiveness or symptoms associated with such tumor growth comprising administering an effective amount of basiliximab to a patient in need thereof.

9. A method of immunotherapy comprising administering to a patient in need thereof an effective amount of basiliximab, optionally in combination with at least one anti-proliferative agent, chemotherapeutic agent or anti-infectious agent.

10. A method according to claim 9, wherein the immunotherapy is preventing or treating atherosclerosis, carcinomas, thrombosis, restenosis, sclerodermitis, fibrosis or for treating solid tumor invasiveness or symptoms associated with such tumor growth.

11. The method of claim 4, wherein the heparanase inhibitor is PI-88, wherein the biological response modifier is a lymphokine or an interferon, wherein the Ras oncogenic isoform is H-Ras, K Ras or N-Ras, wherein the farnesyl transferase inhibitor is 744,832 or DK8G557, wherein the telomerase inhibitor is telomestatin, wherein the aminopeptidase inhibitor is bengamide or a derivative thereof, and wherein the proteosome inhibitor is PS-341.

12. The method of claim 6, wherein the proliferative disease is cancer.

13. The method of claim 6, wherein the infectious disease is microbial infection.

14. The method of claim 7, wherein the immunotherapy is cancer immunotherapy.

15. The method of claim 7, wherein the effective amount is sufficient to inhibit, block or inactivate regulatory T-cells

16. The method of claim 9, wherein the immunotherapy is for the prevention or treatment of a proliferative disease or an infectious disease.
